SEQUENCE LISTING (10 sequences)

SEQ ID NO: 1 - DNA, 27 bp, Artificial Sequence
  biomarker for detecting F06
agagatgaggagcttgctcttcaaatc

SEQ ID NO: 2 - DNA, 35 bp, Artificial Sequence
  biomarker for detecting F06
acggctcggcatcgagcagagggaaaaggagtgat

SEQ ID NO: 3 - DNA, 27 bp, Artificial Sequence
  F06 V1
agagatgargagyttgctcttcaratc

SEQ ID NO: 4 - DNA, 35 bp, Artificial Sequence
  F06 V2
acrgstcggcatcgascagrgggaaaaggagyrat

SEQ ID NO: 5 - DNA, 199 bp, Fusobacterium prausnitzii
cgaacgagagatgaggagcttgctcttcaaatcgagtggcgaacgggtgagtaacgcgtgaggaacctgcctgaaagagggggacaacagttggaaacgactgctaataccgcataagcccacggctcggcatcgagcagagggaaaaggagcgatccgctttgagatggcctcgcgtccgattagctagttggtgagg

SEQ ID NO: 6 - DNA, 199 bp, Fusobacterium prausnitzii
cgaacgagcgagagagagcttgctttctcgagcgagtggcgaacgggtgagtaacgcgtgaggaacctgcctcaaagagggggacaacagttggaaacgactgctaataccgcataagcccacagctcggcatcgagcagagggaaaaggagcaatccgctttgagatggcctcgcgtccgattagctagttggtgagg

SEQ ID NO: 7 - DNA, 199 bp, Fusobacterium prausnitzii; identical to SEQ ID NO: 5

SEQ ID NO: 8 - DNA, 199 bp, Fusobacterium prausnitzii
  n (15)..(15): “N” represents unknown nucleotide
  misc_feature (15)..(15): n is a, c, g, or t
cgaacgagcgagagngaggttgctttctcgagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgagg

SEQ ID NO: 9 - DNA, 55 bp, Artificial Sequence
  forward primer for PCR
  n (26)..(33): “N” represents any base selected from A, G, C or T.
  misc_feature (26)..(33): n is a, c, g, or t
cgtatcgcctccctcgcgccatcagnnnnnnnntcagagtttgatcctggctcag

SEQ ID NO: 10 - DNA, 54 bp, Artificial Sequence
  reverse primer for PCR
  n (26)..(33): “N” represents any base selected from A, G, C or T.
  misc_feature (26)..(33): n is a, c, g, or t
ctatgcgccttgccagcccgctcagnnnnnnnncatgctgcctcccgtaggagt

(19) United States
(12) Patent Application Publication
(10) Pub. No.: US 2022/0170080 A1
(43) Pub. Date: Jun. 2, 2022

(54) BIOMARKER COMPOSITION, DIAGNOSTIC KIT, AND METHOD FOR PROVIDING INFORMATION

(71) Applicant: KOREA UNIVERSITY RESEARCH AND BUSINESS FOUNDATION, Seongbuk-gu Seoul (KR)

(72) Inventor: Hee Nam KIM, Yeongdeungpo-gu Seoul (KR)

(21) Appl. No.: 17/675,589

(22) Filed: Feb. 18, 2022

Related U.S. Application Data
(62) Division of application No. 15/762,441, filed on Mar. 22, 2018.

(30) Foreign Application Priority Data
Sep. 22, 2015 (KR) 10-2015-0134067
Sep. 22, 2015 (KR) 10-2015-0134129

(51) Int. Cl. C12Q 1/689

(52) U.S. Cl. CPC C12Q 1/689; C12Q 2600/156; C12Q 2600/118; C12Q 2600/112

(57) ABSTRACT

Provided are a biomarker composition that includes a nucleotide with a nucleotide sequence of SEQ ID NO: 1 or SEQ ID NO: 2 as an active ingredient, a diagnostic kit that includes the biomarker composition, and a method of providing information for diagnosis of atopic dermatitis (AD) that includes detecting a subspecies of F. prausnitzii (F06). By using the present disclosure, whether a subspecies of F. prausnitzii (F06) having a specific nucleotide sequence is present may be usefully determined, and in particular, whether AD is likely to occur may be easily and accurately diagnosed before AD occurs, and thus the present disclosure may be very useful for individuals both before and after AD occurs.

### CROSS-REFERENCE TO RELATED APPLICATIONS

The present application is a divisional of U.S. patent application Ser. No. 15/762,441 filed Mar. 22, 2018, which is a 371 National Entry Application of PCT/KR2016/010511 filed Sep. 21, 2016 which claims priority to South Korean Application No. 10-2016-0120005, filed Sep. 20, 2016, South Korean Application No. 10-2015-0134129, filed Sep. 22, 2015, and South Korean Application No. 10-2015-0134067, filed Sep. 22, 2015, the contents of which are herein incorporated in their entireties.

### SEQUENCE LISTING

The text of the computer readable sequence listing filed herewith, titled “35654-402_ST25”, created Feb. 18, 2022, having a file size of 4,000 bytes, is hereby incorporated by reference in its entirety.

### TECHNICAL FIELD

The following description relates to a biomarker composition that includes a nucleotide sequence of SEQ ID NO: 1 or SEQ ID NO: 2 as an active ingredient, a diagnostic kit that includes the biomarker composition, and an information providing method that includes an operation of recognizing a nucleotide sequence including SEQ ID NO: 1 or SEQ ID NO: 2.

### BACKGROUND ART

Atopic dermatitis (AD) is a chronic inflammatory skin disorder, and its incidence is increasing globally. AD is a chronic recurrent inflammatory skin disorder that mostly starts in infancy or childhood, and is accompanied by pruritus, xeroderma, or severe inflammation.

Although symptoms of AD vary with age, lesions are often accompanied by pruritus. In early childhood, the symptoms may appear on cheeks, foreheads, heads, trunks, arms or legs. In late childhood, symptoms mainly appear on intertriginous areas of arms, legs or earlobes.

In the United States, up to 25% of children and 2% to 3% of adults suffer from AD. However, causes of AD are not yet known. Although it is not possible to accurately explain the causes, environmental factors or genetic factors are regarded as major causes in view of the fact that cases are increasing mainly in countries industrialized in recent years.

There is no specific test for diagnosis of AD, however, tests to identify factors that aggravate AD are available. Tests to identify allergens that cause AD may include skin prick tests, serum-specific immunoglobulin E tests, oral food challenge tests, and bacterial culture tests.

Although the immediate cause and pathogenesis of AD are still unclear, skin damage in patients is regarded to be primarily caused through a mechanism involving the overproduction of proinflammatory cytokines that cause inflammation when a TH 2 type immune response reacts in a hypersensitive manner to a general antigen. Moreover, other characteristics, such as simplification of the microbial community on the skin and an increase in Staphylococcus aureus, have been observed. It has also been reported that specific bacteria in the intestines may be associated with an atopic disease.

Despite the above research results, the microbiological cause of the dysregulation of inflammation that is the basis of atopy remains unknown. Thus, an important issue in this field is to be able to determine the biological cause of atopy and to diagnose it to clarify that it has indeed developed.

### DISCLOSURE OF INVENTION

### Technical Subjects

According to an aspect, example embodiments provide a biomarker composition including a nucleotide sequence of SEQ ID NO: 1 or SEQ ID NO: 2 as an active ingredient.

Example embodiments provide a biomarker composition for detecting a subspecies of Faecalibacterium prausnitzii (F06).

Example embodiments provide a biomarker composition for diagnosis of atopic dermatitis (AD).

Example embodiments provide a biomarker composition including a nucleotide sequence having a sequence identity of 97% or higher to SEQ ID NO: 1 or SEQ ID NO: 2 as an active ingredient.

According to another aspect, example embodiments provide a diagnostic kit including a biomarker composition including a nucleotide sequence of SEQ ID NO: 1 or SEQ ID NO: 2 as an active ingredient.

Example embodiments provide a diagnostic kit including a biomarker composition for detecting a subspecies of F. prausnitzii (F06), and more particularly, a diagnostic kit including a biomarker composition for diagnosis of AD.

Example embodiments provide a diagnostic kit including a biomarker composition including a nucleotide sequence having a sequence identity of 97% or higher to SEQ ID NO: 1 or SEQ ID NO: 2 as an active ingredient.

According to another aspect, example embodiments provide a method of providing information, the method including providing an isolated nucleic acid sample; and recognizing a nucleotide sequence including SEQ ID NO: 1 or SEQ ID NO: 2.

The recognizing of the nucleotide sequence including SEQ ID NO: 1 or SEQ ID NO: 2 may include detecting a subspecies of F. prausnitzii (F06).

The recognizing of the nucleotide sequence including SEQ ID NO: 1 or SEQ ID NO: 2 may be performed to diagnose AD.

The nucleotide sequence including SEQ ID NO: 1 or SEQ ID NO: 2 may be a nucleotide sequence having a sequence identity of 97% or higher to SEQ ID NO: 1 or SEQ ID NO: 2.

### Technical Solutions

Hereinafter, example embodiments will be described in detail with reference to the accompanying drawings, and like reference numerals in the drawings refer to like elements throughout.

Various modifications may be made to the following example embodiments. Here, the example embodiments are not construed as limited to the disclosure and should be understood to include all changes, equivalents, and replacements within the idea and the technical scope of the disclosure.

The terminology used herein is for the purpose of describing particular example embodiments only and is not to be limiting of the examples. As used herein, the singular forms are intended to include the plural forms as well, unless the context clearly indicates otherwise. It should be further understood that the terms “include/comprise” and/or “have” when used in this specification, specify the presence of stated features, integers, steps, operations, elements, components and/or combinations thereof, but do not preclude the presence or addition of one or more other features, integers, steps, operations, elements, components, and/or groups thereof.

Unless otherwise defined, all terms including technical and scientific terms used herein have the same meaning as commonly understood by one of ordinary skill in the art to which example embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

Also, in the following description with reference to the accompanying drawings, like reference numerals refer to like constituent elements, and a repeated description related thereto will be omitted. When it is determined detailed description related to a related known function or configuration they may make the purpose of the example embodiments unnecessarily ambiguous in describing the example embodiments, the detailed description will be omitted here.

Hereinafter, the terms used in the present disclosure are described.

The term “biomarker,” as used herein, generally refers to an indicator to identify a change in a body using proteins, DNA, RNA, metabolites, and the like.

The term “genome,” as used herein, refers to a cluster of genes that include all genetic information for a human body, and the term “microbiome,” which is called the “second genome,” refers to all genetic information of microorganisms coexisting in the body. In particular, the term “gut microbiome” refers to all genetic information of microorganisms existing in the body, in particular, the intestines. Recently, the interest in the gut microbiome has increased because it has been reported that various microorganisms in the human body influence metabolism regulation, digestive ability, or various diseases as well as influence all functions of the human body such as genetic modifications due to environmental changes.

The term “diagnosis,” as used herein, may include a prediction or diagnosis of the outcome or state of an individual, the state or outcome, the progression of a condition, or a prediction of the response to a specific treatment. In example embodiments of the present disclosure, typical technologies of immunology, biochemistry, molecular biology, microbiology, cell biology, genomics, and recombinant DNA within the skill of the related art may be employed, unless otherwise indicated. The terms “individual,” “patient,” or “object,” as used herein, may include humans and other mammals.

Throughout the present specification and drawings, the term “AD” indicates an atopic dermatitis, the term “SCFA” indicates short-chain fatty acid, the term “OTU” indicates operational taxonomic unit, the term “GalNAc” indicates N-acetylgalactosamine, and the term “KO” indicates Kyoto Encyclopedia of Genes and Genomes (KEGG) Orthology.

According to an aspect, a biomarker composition including a nucleotide sequence of SEQ ID NO: 1 or SEQ ID NO: 2 as an active ingredient may be provided.

The biomarker composition including the nucleotide sequence of SEQ ID NO: 1 or SEQ ID NO: 2 as an active ingredient may be used to determine the presence or absence of various diseases including the nucleotide sequence of SEQ ID NO: 1 or SEQ ID NO: 2 from samples of various individuals with a disease or suspected of having a disease.

The nucleotide sequences of SEQ ID NO: 1 and SEQ ID NO: 2 are provided below (see FIGS. 4A through 4D).

SEQ ID NO: 1
AGAGATGAGGAGCTTGCTCTTCAAATC
SEQ ID NO: 2
ACGGCTCGGCATCGAGCAGAGGGAAAAGGAGTGAT

Referring to FIG. 4B, a nucleotide sequence of an F06 V1 region is not limited to SEQ ID NO: 1 and may have G or A at position 9, C or T at position 13, and A or G at position 24 ((position 9(G, A), position 13(C, T), position 24(A, G)). In FIG. 4B, a nucleotide sequence of an F06 V2 region is not limited to SEQ ID NO: 2 and may have G or A at position 3, C or G at position 5, G or C at position 16, A or G at position 20, T or C at position 32, and G or A at position 33 (position 3(G, A), position 5(C, G), position 16(G, C), position 20(A, G), position 32(T, C), position 33(G, A)).

For example, the biomarker composition may be a biomarker composition for detecting a subspecies of F. prausnitzii (F06).

The nucleotide sequence of SEQ ID NO: 1 or SEQ ID NO: 2 may be used as a biomarker composition for detecting a subspecies of F. prausnitzii (F06) in a microbiome. A biomarker composition including the nucleotide sequence may be used to diagnose a variety of diseases related to the subspecies of F. prausnitzii (F06).

As another example, the biomarker composition may be a biomarker composition for diagnosing AD.

Based on the V1-V2 sequence indicating the nucleotide sequence of SEQ ID NO: 1 or SEQ ID NO: 2, a great increase in the AD microbiome was confirmed (see FIGS. 3A through 3C). In particular, an increase in F06 in the AD microbiome was observed to be common for all ages but was especially prominent in the age group that was less than one year old, which indicates that the subspecies of F. prausnitzii (F06) is involved in an onset of AD. The biomarker composition was determined to be suitable for the diagnosis of AD, as described above, and may also be used to diagnose all diseases involving the nucleotide sequence of SEQ ID NO: 1 or SEQ ID NO: 2.

According to another aspect, the biomarker composition may be a biomarker composition including a nucleotide sequence having a sequence identity of 97% or higher to SEQ ID NO: 1 or SEQ ID NO: 2 as an active ingredient.

Actually, among Koreans, differences have been found in the nucleotides at positions 9, 13, and 24 of SEQ ID NO: 1 and positions 3, 5, 16, 20, 32, and 33 of SEQ ID NO: 2. Thus, each of the nucleotides at positions 9, 13, and 24 of SEQ ID NO: 1 and positions 3, 5, 16, 20, 32, and 33 of SEQ ID NO: 2 may be variously replaced. Desirably, SEQ ID NO: 1 or SEQ ID NO: 2 may be replaced with one having a sequence identity of 97% or higher to the entire sequence. For example, G at position 9 of SEQ ID NO: 1 may be replaced with A, C at position 13 may be replaced with T, and A at position 24 may be replaced with G. In addition, G at position 3 of SEQ ID NO: 2 may be replaced with A, C at position 5 may be replaced with G, G at position 16 may be replaced with C, A at position 20 may be replaced with G, T at position 32 may be replaced with C, and G at the position 33 may be replaced with A. In other words, the biomarker composition may include, for example, a biomarker composition including a nucleotide sequence having a sequence identity that is 97% or higher to SEQ ID NO: 1 or SEQ ID NO: 2 as an active ingredient so that it is within the range of the homogeneous diversity of 16S rRNA genes of typical bacteria.

According to another aspect, a diagnostic kit including a biomarker composition that includes a nucleotide sequence of SEQ ID NO: 1 or SEQ ID NO: 2 as an active ingredient may be provided.

A diagnostic kit for diagnosis of a composition including a nucleotide sequence of SEQ ID NO: 1 or SEQ ID NO: 2, and desirably, a diagnostic kit for diagnosis of AD may be provided when a nucleic acid sample isolated from an individual is provided. The diagnostic kit may be used for any type of disease related to the subspecies of F. prausnitzii (F06). Moreover, the diagnostic kit, after being determined to be suitable for the diagnosis of AD, may also be used to diagnose all diseases involving the nucleotide sequence of SEQ ID NO: 1 or SEQ ID NO: 2.

In addition, a diagnostic kit including a biomarker composition including a nucleotide sequence having a sequence identity of 97% or higher to SEQ ID NO: 1 or SEQ ID NO: 2 as an active ingredient may be provided.

According to another aspect, a method of providing information may be provided. The method may include providing an isolated nucleic acid sample, and recognizing a nucleotide sequence that includes SEQ ID NO: 1 or SEQ ID NO: 2.

When a nucleic acid sample isolated from an individual is provided, a nucleotide sequence including SEQ ID NO: 1 or SEQ ID NO: 2 may be recognized, and useful information required for an early diagnosis and prognosis of various diseases, and desirably, useful information required for an AD diagnosis may be provided.

The recognizing of the nucleotide sequence including SEQ ID NO: 1 or SEQ ID NO: 2 may include detecting a subspecies of F. prausnitzii (F06).

A method of providing information for detection of a subspecies of F. prausnitzii (F06) by recognizing a nucleotide sequence including SEQ ID NO: 1 or SEQ ID NO: 2 when a nucleic acid sample isolated from an individual is provided, may be provided, and in particular, a method of providing information for diagnosis of AD associated with the above bacteria may be provided.

The nucleotide sequence including SEQ ID NO: 1 or SEQ ID NO: 2 may be a nucleotide sequence having a sequence identity of 97% or higher to SEQ ID NO: 1 or SEQ ID NO: 2.

Actually, among Koreans, differences have been found in nucleotides at positions 9, 13, and 24 of SEQ ID NO: 1 and positions 3, 5, 16, 20, 32, and 33 of SEQ ID NO: 2. Thus, each of the nucleotides at positions 9, 13, and 24 of SEQ ID NO: 1 and positions 3, 5, 16, 20, 32, and 33 of SEQ ID NO: 2 may be variously replaced. Desirably, SEQ ID NO: 1 or SEQ ID NO: 2 may be replaced with one having a sequence identity of 97% or higher to the entire sequence. For example, G at position 9 of SEQ ID NO: 1 may be replaced with A, C at position 13 may be replaced with T, and A at position 24 may be replaced with G. In addition, G at position 3 of SEQ ID NO: 2 may be replaced with A, C at position 5 may be replaced with G, G at position 16 may be replaced with C, A at position 20 may be replaced with G, T at position 32 may be replaced with C, and G at position 33 may be replaced with A. In other words, the biomarker composition may include, for example, a biomarker composition including a nucleotide sequence having a sequence identity that is 97% or higher to SEQ ID NO: 1 or SEQ ID NO: 2 as an active ingredient so that it is within the range of the homogeneous diversity of 16S rRNA genes of typical bacteria.

### Effect of the Invention

According to example embodiments, a biomarker composition that includes a nucleotide sequence of SEQ ID NO: 1 or SEQ ID NO: 2 as an active ingredient, and a diagnostic kit that includes the biomarker composition may be effectively used for diagnosis and treatment of atopic dermatitis (AD) with a high incidence worldwide.

According to example embodiments, whether a subspecies of F. prausnitzii (F06) having a specific nucleotide sequence is present may be usefully determined, and in particular, whether AD is likely to occur may be easily and accurately diagnosed before AD occurs, and thus the present disclosure may be very useful for individuals both before and after AD occurs.

### BRIEF DESCRIPTION OF DRAWINGS

FIG. 1A illustrates an a diversity profile of a gut microbiome, FIG. 1B illustrates a (3 diversity profile of the gut microbiome, FIG. 1C illustrates an analysis of enterotypes, and FIG. 1D illustrates a relative abundance (%) of Ruminococcus, Parabacteroides, Faecalibacterium and Escherichia/Shigella.

FIG. 2A is a histogram showing results from an RFE-SVM analysis, and FIG. 2B illustrates a list of bacterial clades including top discriminatory OTUs.

FIG. 3A illustrates a phylogenetic tree of F. prausnitzii OTUs to select operational taxonomic units (OTUs) for distinguishing between atopic patients and non-atopic controls, FIG. 3B illustrates an OUT abundance, and FIG. 3C illustrates an F06 abundance.

FIG. 4A illustrates a V1-V2 region of a 16S rRNA gene of each F. prausnitzii strain (SEQ ID NOs: 6-8), FIG. 4B illustrates a consensus sequence of an F06 V1 region (SEQ ID NO: 3) and F06 V1 region (SEQ ID NO: 4), FIG. 4C illustrates all F06 454 reads, and FIG. 4D illustrates a relative abundance of a V1 sequence.

FIG. 5A illustrates a heat map of significantly altered functions in an atopic dermatitis (AD) microbiome, and FIG. 5B illustrates sequences of genes of five strains of F. prausnitzii, that is, A2-165, M21/2, L2-6, S3L/3 and KLE1255.

FIG. 6A is a graph illustrating analyses of SCFAs of an experimental group and a control group, and FIG. 6B illustrates a comparison of butyryl CoA:acetate CoA-transferase gene promoter activity from F. prausnitzii strains.

FIG. 7 illustrates a proposed model of AD associated with a dysfunction of F. prausnitzii.

FIG. 8A is a Venn diagram illustrating common genes or unique genes in strains, and FIG. 8B illustrates Illumina reads of each gene group.

### MODE FOR CARRYING OUT THE INVENTION

Hereinafter, the present disclosure will be described in detail based on example embodiments.

However, the following example embodiments are merely examples, and the present disclosure is not limited to the following example embodiments.

The inventor of the present disclosure has estimated that specific bacteria in the intestines may be associated with an atopic disease and has studied the correlation between intestinal bacteria and atopy for a long period of time. To demonstrate the correlation, “90” atopic patients (experimental group) and “42” subjects without atopic symptoms (control group) participated in the present study, and the 16S rRNA genes and metagenomes of the gut microbial community of “132” subjects in total were analyzed. Reference gene data from the Human Microbiome Project and the KEGG Orthology database were used for the metagenomic analysis. The severity of the symptoms of atopic patients was evaluated in advance using a SCORAD system, and the control group was formed of individuals who never had atopic dermatitis (AD). Both the experimental group and the control group were not exposed to antibiotics within the six months prior to providing fecal samples. The experimental group was composed of outpatients at the Anam Hospital of Korea University, and the present study was approved by the Ethics Committee. Fecal samples of approximately 5 g were obtained from the experimental group and the control group and stored at −80° C. until DNA was isolated. Short-chain fatty acids (SCFAs) in the fecal samples were compared and analyzed using a gas chromatographic mass spectrometer.

### Example 1: DNA Isolation

Samples were ground in liquid nitrogen with a mortar and pestle, and 400 mg of the ground samples was transferred to four microcentrifuge tubes. Each of the tubes contained 500 μl of a solution including 0.1 mm-diameter zirconia/silica beads (BioSpec products, Bartlesville, Okla., USA), 500 μl of an extraction buffer (200 mM Tris [pH 8.0], 200 mM NaCl, 20 mM EDTA), 210 μl of 20% SDS, and a phenol:chloroform:isoamyl alcohol mixture (25:24:1, pH 7.9).

The samples were ground with a bead beater for 2.5 minutes at room temperature, and the bacterial contents were released by disrupting bacterial cell envelopes. DNA was isolated from the supernatant using the phenol/chloroform/isoamyl alcohol (25:24:1, pH 7.9) and precipitated with isopropanol. DNA pellets were dried and dissolved in a TE buffer (10 mM Tris [pH 8.0], 1 mM EDTA), and the concentration was adjusted to 100 ng/μl.

### Example 2: Sequence Analysis of V1-V2 Region of 16S rRNA Gene

A PCR reaction targeting the V1-V2 region of the 16S rRNA gene was conducted with each DNA sample in a 25-μl reaction mixture including 100 ng of template DNA, 5 μl of a HotStar PCR buffer (Qiagen, Germantown, Md., USA), 1 U of HotStarTaq DNA polymerase (Qiagen), and 0.4 μmol/l each of a forward primer and a reverse primer. The forward primer, 5′-CGTATCGCCTCCCTCGCGCCATCAGNNNNNNNNTCAGAGTTTGATCCTGGCTCAG-3′ (SEQ ID NO: 9), was formed of a 454 titanium adapter A (underlined nucleotides), a unique 8-base barcode (shown with eight Ns), a linker (a dinucleotide TC), and universal bacterial primer 8F. The reverse primer, 5′-CTATGCGCCTTGCCAGCCCGCTCAGNNNNNNNNCATGCTGCCTCCCGTAGGAGT-3′ (SEQ ID NO: 10), was formed of a 454 titanium adapter B (underlined), a unique 8-base barcode (Ns), a linker (CA), and a broad-range bacterial primer 338R (in italics).

A DNA amplification was conducted at 95° C. for 15 minutes, followed by 34 cycles of 95° C. for 30 seconds, 52° C. for 30 seconds, and 72° C. for 1 minute, with a final extension at 72° C. for 5 minutes. PCR products were obtained using a QIAquick PCR Purification Kit (Qiagen). Amplicons were quantified with the PicoGreen Assay and diluted to 1×10⁹ molecules/μl in the TE buffer. A sequencing library was constructed for 454FLX titanium pyrosequencing using an equal amount of each product.

### Example 3: Analysis of 454 Sequencing Reads

Barcode-tagged V1-V2 region sequence reads of the 16S rRNA gene were analyzed using a QIIME pipeline. Sequences lacking either the 5′ or 3′ end sequences, or sequences including at least one ambiguous nucleotide were excluded.

All primer sequences were removed and trimmed sequences shorter than 250 bp were excluded. The other sequences were classified into operational taxonomic units (OTUs) using a CD-HIT based on a 97% sequence similarity and were taxonomically identified using an RDP classifier based on a confidence score of 0.5%. Chimera sequences were removed using a ChimeraSlayer and a BlastFragment in the QIIME pipeline. The Shannon index was calculated to know the α-diversity of the gut microbiota, and an unweighted UniFrac or Bray-Curtis distance metrics for a β-diversity analysis were constructed using QIIME scripts.

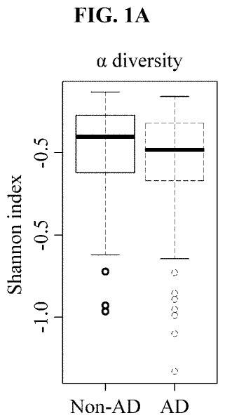

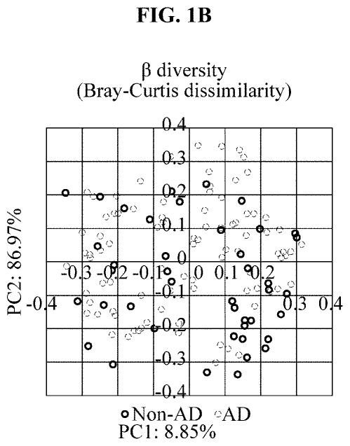

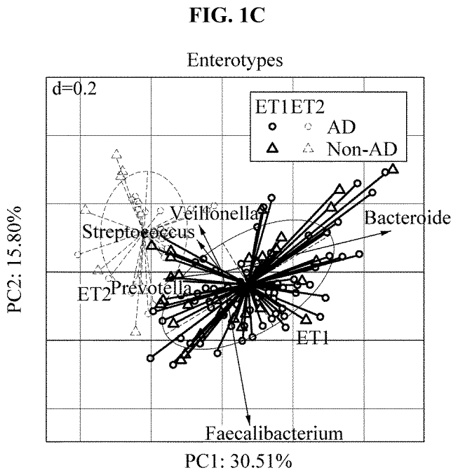

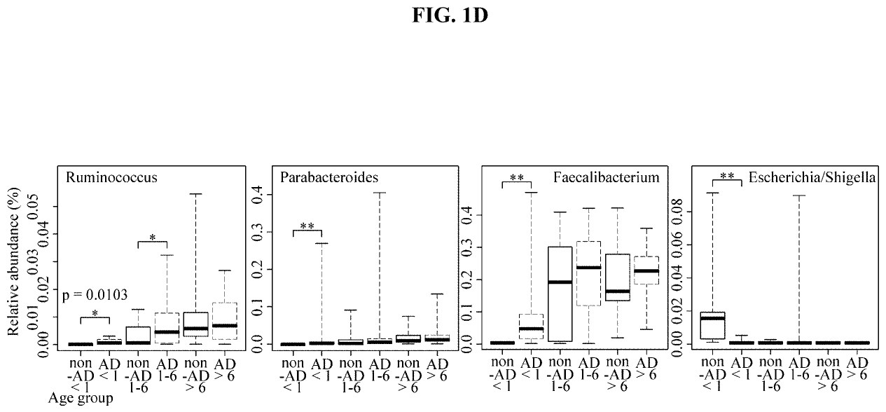

An enterotype analysis used a genus-level distance matrix constructed based on the Jensen-Shannon divergence algorithm, and was conducted using the partitioning around medoids (PAM) clustering algorithm. The Calinski-Harabasz index was calculated to know the optimal number of an enterotype cluster (see FIGS. 1A through 1D).

454 sequencing reads were analyzed through an OTU analysis using the CD-HIT or analyzed by constructing taxon-independent OTUs using a separate ESPRIT. An OTU abundance matrix formed by the above OTUs was used to identify the most discriminatory OTU between atopic patients and non-atopic controls using a recursive feature elimination support vector machine (RFE-SVM). OTUs that were absent in at least 30% of samples were excluded from data to reduce the noise level. A leave-one-out cross-validation was used to measure the prediction accuracy for the identification of the AD-microbiota using a selected set of discriminatory OTUs.

### Example 4: Whole-Genome Sequencing and Sequence Analysis

Whole-genome sequences of eight fecal samples (four samples each from the experimental group and the control group) were obtained using the Illumina HiSeq2000 platform. A DNA library with a fragment length of approximately 400 bp was prepared, and paired-end reads of 101 bp were obtained.

Sequence analysis was conducted with a CLC Genomics Workbench. Low-quality reads were excluded based on the following: all sequences with a quality score less than 0.05 or a sequence with at least two ambiguous nucleotide sequences. Sequences shorter than 15 bp were also excluded. Functions of the metagenome were analyzed using HUMAnN v0.99. All Bacteroides and Faecalibacterium genomes (bfg, bhl, bsa, bxy, bdo, bdh, bacc, fpr, and fpl) were added to a KEGG Orthology (KO) database v54 and used for analysis. A translated BLAST search of the Illumina reads against the KO database was conducted using USEARCH v8.0. Results were processed with HUMAnN, and normalized orthologous gene family abundances were compared between the experimental group and control group.

### Example 5: Pangenomic Analysis of Five F. prausnitzii Strains

Genome sequences of five F. prausnitzii strains were downloaded from the Human Microbiome Project (HMP) website (http://www.hmpdacc.org/) to perform a pangenomic analysis. Coding DNA sequences (CDSs) from the strains were compared using BLASTP and clustered into discrete orthologs based on amino acid level similarity and matching regions of 70% or greater. The CDSs were organized using the KO database based on BLASTP hits, with an e-value cutoff of 1e-5.

Whole-metagenome reads from Illumina HiSeq2000 sequencing were mapped on the genomes of the five F. prausnitzii strains using CLC Genomics Workbench. As a result, 10.14% of the Illumina reads were mapped on the F. prausnitzii genomes.

### Example 6: Measurement of SCFAs in Samples

Samples were ground in liquid nitrogen with a mortar and pestle, and approximately 0.1 g of the ground samples was transferred to a 2-mL microfuge tube. An internal standard (60 μL of 0.25 mmol/l 4-methylvaleric acid) was added to a tube including a ground sample and used to measure SCFAs. After acidification of the samples with 20 μL of 33% hydrogen chloride (HCl), diethyl ether was added, followed by vortexing for 10 minutes. The diethyl ether layer was centrifuged and transferred to a new tube. The same procedure was repeated one more time, and then, the diethyl ether phases from two extractions were mixed. 60 μl of the extract and 20 μL of N-tert-butyldimethylsilyl-N-methyltrifluoroacetamide were mixed in a glass insert in a gas chromatography autosampler vial and incubated for 2 hours at room temperature. Gas chromatography mass spectrometry was conducted using the prepared samples. Ratios of D-lactate and L-lactate were acquired using a Megazyme enzymatic kit (Megazyme, Ireland).

Frozen samples (0.1 g) were dissolved in 1 ml of 0.1 M triethanolamine buffer (pH 9.15) and centrifuged at 14,000 for 10 minutes at 4° C. The supernatant was precipitated with 6.1 N trichloroacetic acid (10% final concentration) and centrifuged at 4500 g for 20 minutes at 4° C. The precipitated supernatant was used to measure a the lactate level.

### Example 7: Promoter Activity Analysis

To compare the promoter activity of the butyryl CoA:acetate CoA-transferase gene in five F. prausnitzii strains (FP2_20620 for strain L2-6, FAEPRAA2165_01575 for strain A2-165, HMPREF9436_00973 for strain KLE1255, FPR 29560 for strain SL3/3, and FAEPRAM212_02812 for strain M21/2), a lacZY fusion was made to the promoter of each gene using the promoter probe vector pLKC481, and plasmid constructs were transferred to Escherichia coli DH5a. E. coli strains including different pLKC481 constructs were grown in an LB broth to the mid-log phase (OD₆₀₀=0.7) and left on ice for 20 minutes. 2 ml of each sample was harvested, and pellets were resuspended in a Z buffer. A β-galactosidase activity representing the promoter activity was measured using O-nitrophenyl-β-D-galactoside as a substrate and expressed as Miller units calculated using the following expression:

1000×(OD₄₂₀−1.75×OD₅₅₀)/(Time of incubation [min]×Volume [ml]×OD₆₀₀)

Hereinafter, the present disclosure will be further described based on the drawings.

FIG. 1A illustrates an a diversity profile of a gut microbiome, FIG. 1B illustrates a β diversity profile of the gut microbiome, FIG. 1C illustrates an analysis of enterotypes, and FIG. 1D illustrates a relative abundance (%) of Ruminococcus, Parabacteroides, Faecalibacterium and Escherichia/Shigella.

DNA representing the gut microbiome from “90” subjects in the experimental group and “43” subjects in the control group was isolated from fecal samples, and V1-V2 region in 16S rRNA genes were amplified and sequenced using 454 technology. The sequence reads were found to have an average sequencing depth of 6,604 reads after low-quality sequences were removed. No notable difference was found in a microbial a diversity between the microbiomes of atopic patients and those of non-atopic controls (see a graph of FIG. 1A). Moreover, no notable difference was found in microbial 13 diversity (composition) between the atopic patients and non-atopic controls (see a graph of FIG. 1B). The microbiome may be designated as a specific enterotype based on major bacteria groups. Although the “132” microbiomes were classified as one of two enterotypes, that is, Bacteroides or Prevotella, AD was not classified as one of the enterotypes (see a graph of FIG. 1C). Thus, there was no marked overall change in microbiomes in AD. However, a meaningful difference between the experimental group and the control group was observed for a specific bacterial clade in groups divided by age (see the graph of FIG. 1B). Although there have been reports of an increase in bacteria belonging to the family Enterobacteriaceae and decreases in bacteria belonging to Lactobacillus and Bifidobacterium species in patients with AD, the above trend was not observed in the present experiment. In addition, bacteria reported to be associated with atopy in previous studies may include bacteria with increased opportunistic growth in an unhealthy gut environment and are not directly associated with atopy. Bacteria associated with atopy may include bacteria that directly have pathogenicity or bacteria that simply have a growth advantage in a damaged gut environment. Pathogenic bacteria that play an important role in atopy may be a single species or a consortium of specific bacteria, and may be increased in all atopic environments. Bacteria that simply utilize a disease environment may include various types of bacteria. As a whole, the above bacteria may seem to be increased in atopic environments, however, may be absent in individual samples. Considering the above pattern, most bacteria increased in atopy microbiomes (see the graphs of FIGS. 1A through 1D) are highly likely to be bacteria that simply utilize atopic environments. However, because a few causative bacteria responsible for atopy are present but are mixed with these other bacteria, it may be difficult to distinguish the causative bacteria from the other bacteria due to the overwhelming presence of the other bacteria.

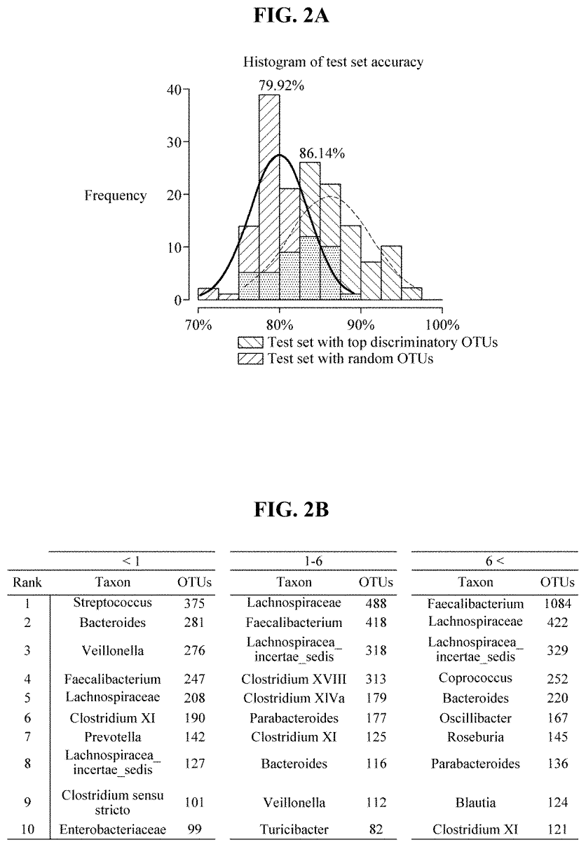

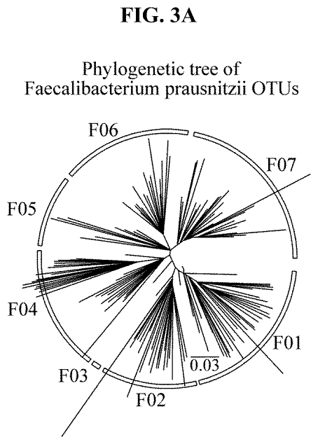

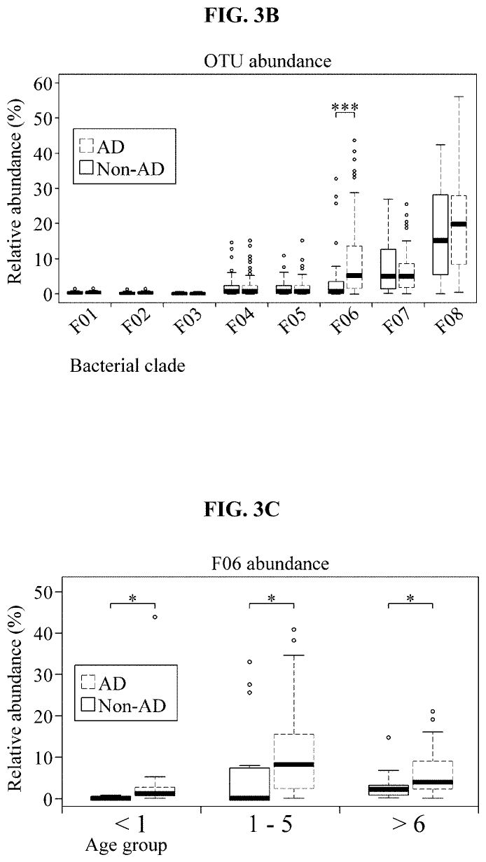

FIG. 2A is a histogram showing results from an RFE-SVM analysis, and FIG. 2B illustrates a list of bacterial clades including top discriminatory OTUs. FIG. 3A illustrates a phylogenetic tree of F. prausnitzii OTUs to select operational taxonomic units (OTUs) for distinguishing between atopic patients and non-atopic controls, FIG. 3B illustrates an OUT abundance, and FIG. 3C illustrates an F06 abundance.

The RFE-SVM analysis was conducted to select the top discriminatory OTUs to distinguish between atopic patients and non-atopic controls. For the above analysis, OTUs were constructed using ESPRIT, which is based on a taxonomy-independent hierarchical classification (at a 0.03 distance level that generally matches the species level). One-half of all data were randomly selected and used as a training set for the RFE-SVM analysis to select discriminatory OTUs. The other half of the data were used to test the atopy identification abilities of the OTUs. The OTUs showed an accuracy of 88.14% in the atopy identification abilities, in comparison to randomly selected OTUs with an accuracy of 79.92% (see FIG. 2A). It was found that a large number of OTUs among the top discriminatory OTUs belonged to a few clades and were common among all ages (see FIG. 2B). Among these, in particular, the Faecalibacterium species was shown to significantly increase in the atopy microbiomes (see the graph of FIG. 1D). Currently, all Faecalibacterium species are grouped into a single species, F. prausnitzii, although two physiologically distinct phylotypes are present.

Atopy-Related Subspecies of F. prausnitzii

F. prausnitzii was markedly present in the atopy microbiome. Because F. prausnitzii is one of most significant species in an RFE-SVM model and is one of most abundant species in the human gut microbiome, an experiment was conducted to determine if it performs an important function in both normal human physiology and AD. Also, because F. prausnitzii is a single species, it was relatively easy to study the composition of the clade. The study was conducted based on the possibility of a specific subspecies being more associated with AD than all species. ESPRIT OTUs annotated as F. prausnitzii were clustered into seven clades in the phylogenetic tree. Because the phylogenetic tree is based on the V1-V2 sequences, it is possible that each clade may be further divided when the entire sequence of the 16S rRNA gene is applied. F06 was one of the clades that was significantly increased in the AD microbiome (see FIGS. 3A through 3C). An increase in F06 in the AD microbiome was common among all ages and was particularly prominent in the less than one-year-old group (see FIGS. 3A through 3C). Because AD mainly starts before one year of age, this result shows that an F. prausnitzii subspecies is involved in the onset of the above disease.

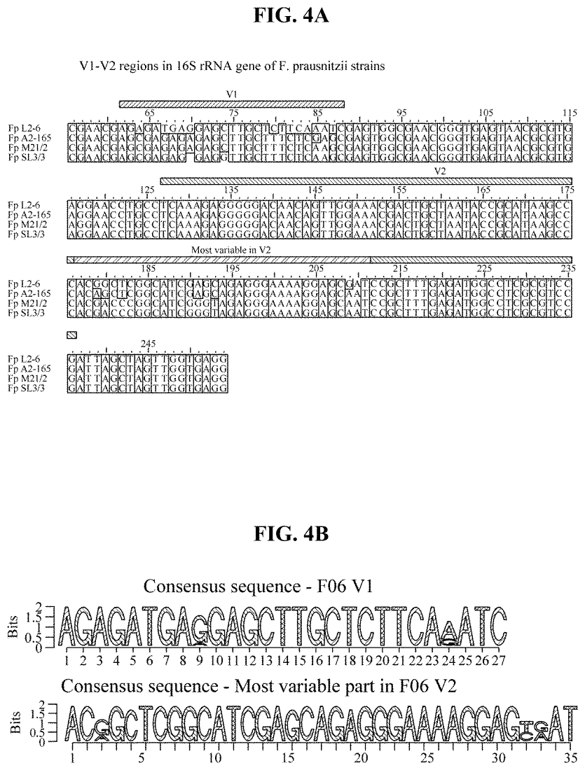

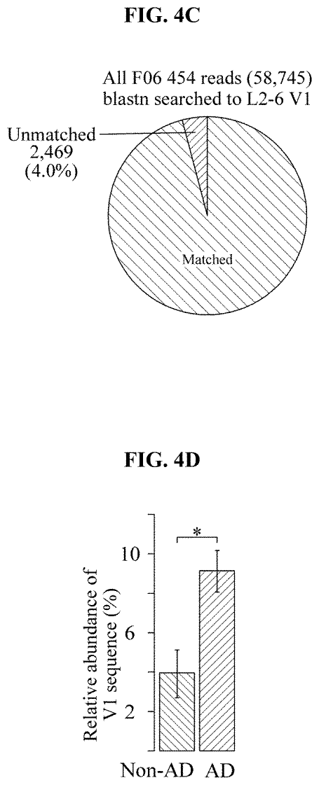

FIG. 4A illustrates the V1-V2 region of the 16S rRNA gene of each F. prausnitzii strain, FIG. 4B illustrates a consensus sequence of the F06 V1 region, FIG. 4C illustrates all F06 454 reads, and FIG. 4D illustrates the relative abundance of the V1 sequence.

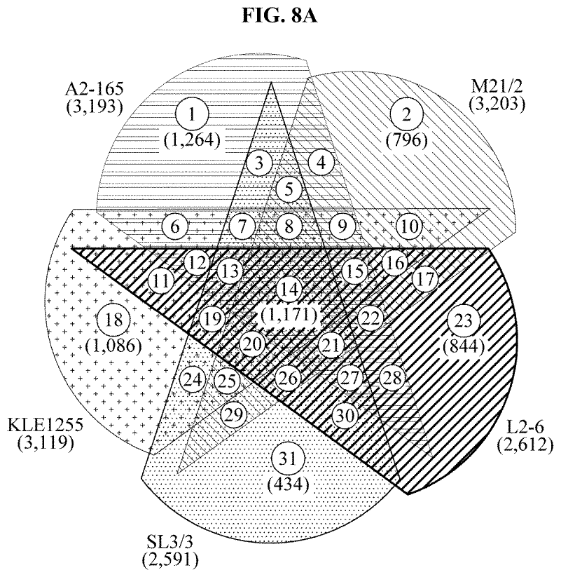

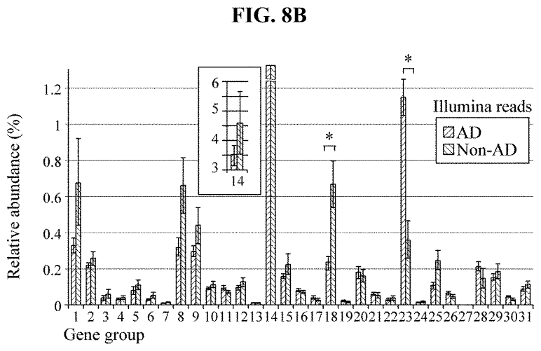

Five strains of F. prausnitzii, that is, A2-165, M21/2, L2-6, S3L/3, and KLE1255, have been sequenced at the genomic level through the Human Microbiome Project (HMP). Among the above strains, L2-6 has a very unique V1-V2 region, in particular, a V1 region, in comparison to those of the four other strains, and closely matches the F06 clade (see FIGS. 4A through 4D). Although the V1-V2 region is a part of the 16S rRNA gene, an almost perfect match over the entire region indicates a very close relationship between L2-6 and F06 bacteria. By performing a cross-comparison of “15,825” predicted protein sequences in the five strains of F. prausnitzii using BLASTP and by removing overlapping genes, it was found that the F. prausnitzii pangenome was formed of “7,253” genes (see FIGS. 8A and 8B). The results indicated that the L2-6 strain had “855” unique genes and that the other strains had a similar number of unique genes. To compare the gut microbiomes and F. prausnitzii pangenome, eight samples (four samples each from the experiment group and the control group) were selected and subjected to whole-genome shotgun sequencing using the Illumina HiSeq 2000 platform, to obtain sequences of 552 Mb on average per sample. The eight samples were selected based on similar and high F. prausnitzii contents (19% to 36%). It was observed that when the Illumina reads were mapped to the F. prausnitzii pangenome, most (87.4%) of genes unique to L2-6 matched the reads from the atopic patients better than the reads from the control group (see FIGS. 8A and 8B). On the other hand, the genes unique to A2-165 and KLE1255 matched the reads from the non-atopic controls much better. Therefore, it was concluded that bacteria belonging to F06 are highly related to the L2-6 strain at the genomic level as well as at the V1-V2 region.

Change in Metabolic Functions of Atopy Microbiome

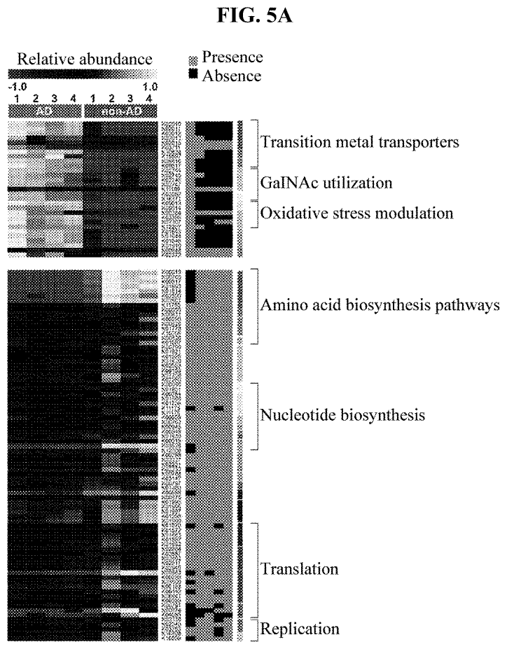

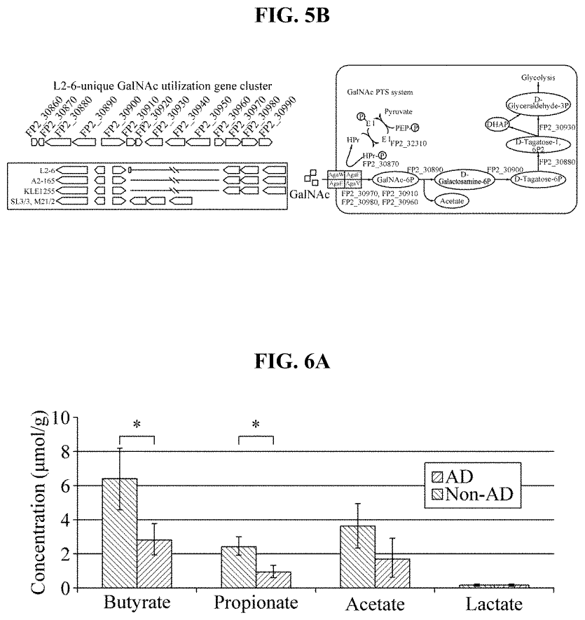

A metabolic change in the AD microbiomes was found using a method of mapping Illumina reads to the F. prausnitzii pangenome and KO database. The F. prausnitzii pangenome approach was effective due to the high F. prausnitzii content (19% to 36%) in the metagenome. Although the F. prausnitzii pangenome is formed of five strains, it is important that two biologically significant strains are included: A2-165, which is known to be lacking in Crohn disease, and L2-6, which was found to be associated with atopy in the present study. To interpret the mapping results, the F. prausnitzii pangenome was analyzed based on the KO database, and KOs (P<0.05) with different numbers of matching reads between the experimental group and control group were collected. Among a total of “1,332” KOs assigned to the pangenome, “415” KOs showed a statistically significant difference between the experimental group and the control group, “122” KOs were increased in the experimental group, and “293” KOs were increased in the control group (see FIGS. 5A and 5B).

KOs that were increased in the AD microbiome include those with metabolic functions responsive to oxidative stress (for example, peroxiredoxin, DMSO reductase, and the sufBC operon), various transition metal transporters (for example, Fe, Ni, Zn, and Mn) that are involved in various pathways, and a function of transporting and performing catabolism of a major mucin component, that is, GalNAc (see FIGS. 5A and 5B). In contrast, genes with a decreased content in the AD microbiome included genes involved in the biosynthesis of various amino acids, nucleotides, peptidoglycans and cofactors (see FIGS. 5A and 5B). In the damaged gut environment, host-derived general nutrients, such as amino acids, cofactors, and coenzymes, as well as specific nutrients such as the mucin component GalNAc may be increased. An increase in general nutrients may result in a reduction in the biosynthetic gene content in the microbiome, and an increase in specific nutrients, such as GalNAc, may result in the selective increase in auxotrophic specialists or pathobionts that are capable of using the above materials. The above gut environment may further promote damage to the gut epidermis, which in turn, accelerates the dysbiosis of the gut microbiome. Among the five strains of F. prausnitzii, only L2-6 contains a gene cluster that utilizes GalNAc (see FIGS. 5A and 5B).

In the second method of mapping the Illumina reads to the KO database, “122” KOs among a total of “5,832” KOs showed differences between the atopy metagenome of the experimental group and metagenome of the control group. “66” KOs and “56” KOs were increased in the atopy metagenome and the metagenome of the control group, respectively. The results of the KO analysis were lower than those of the F. prausnitzii pangenomic analysis; however, this difference was because metagenomes other than F. prausnitzii were included. Similar to the F. prausnitzii pangenomic analysis, the KO analysis showed increases in a metal transporter, use of GalNAc, and oxidative stress response functions, and decreases in the biosynthesis of amino acids, nucleotides, and cofactors in the atopy metagenome. However, an increase in other genes such as genes related to use of L-fucose (L-fuculokinase and L-fucose isomerase) were found. L-fucose is released from mucin glycoproteins and used as a major nutrient component together with GalNAc by gut bacteria such as Bacteroides thetaiotaomicron. It was newly found that genes related to the glutathione S-transferase family, glucose-6-phosphate 1-dehydrogenase, glutamate decarboxylase, and glyoxylate and dicarboxylate metabolism were increased. Glutathione is a tripeptide of cysteine and glutamate and allows bacteria to maintain homeostasis while withstanding oxidative stress caused by reactive oxygen and nitrogen metabolites produced during inflammatory cascades. Glutathione-S-transferases perform a conjugation of a reduced glutathione to an endogenous compound, such as peroxidized lipids, for the purpose of detoxification. Glucose-6-phosphate 1-dehydrogenase levels have been shown to be increased in E. coli under oxidative stress, which leads to increases in antioxidant molecules of glutathione and nicotinamide adenine dinucleotide phosphate. Glutamate decarboxylase has been known to play an important role as an antioxidant in E. coli O157:H7 and Saccharomyces cerevisiae. Also, glyoxylate and dicarboxylate metabolism has been known to be involved in an antioxidation process. Thus, the above gene presence pattern further confirms that the gut epithelium tissues of a patient with AD are heavily damaged by inflammation (see FIGS. 5A and 5B).

A variety of auxotrophs and pathobionts were determined to be increased in the AD microbiome, and a diversity of GalNAc-specific IIA component genes (agaF and K02744) in a PTS system was examined. The results indicate that the above genes exist in a wide variety of forms. The increase in the genes that use mucin in different species indicates that a variety of auxotrophs and pathobionts other than the L2-6 strain flourish in the AD gut environment, which may be associated with the exacerbation and chronic nature of AD.

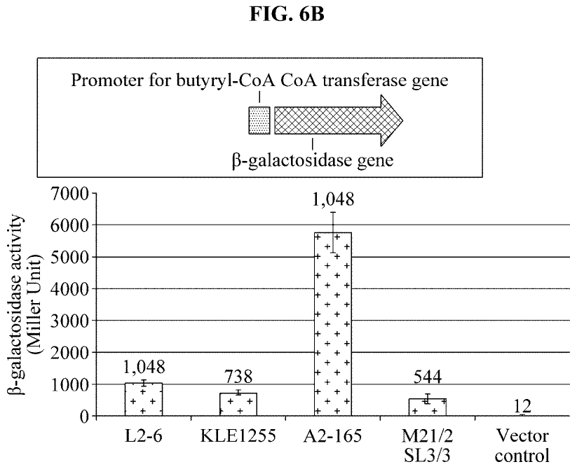

FIG. 6A is a graph illustrating analyses of SCFAs of the experimental group and control group, and FIG. 6B illustrates comparison of butyryl CoA:acetate CoA-transferase gene promoter activity from F. prausnitzii strains.

Increase in F. prausnitzii F06 Subspecies and Reduction in Butyrate and Propionate

The production of SCFAs in the gut microbiome has tremendous health benefits. To verify a difference in the production of SCFAs between the experimental AD group and control group, gas chromatographic-mass spectrometric analyses were conducted (see FIGS. 6A and 6B). Major SCFSs, for example, acetate, propionate, butyrate, and D- and L-lactate, were compared by selecting and pairing samples between the experimental group and control group that had similar total amounts of F. prausnitzii but that had significantly different F06 OTU levels (see FIGS. 6A and 6B). Because the total amount of F. prausnitzii in the samples was much higher than amounts of other SCFA producers, for example, Eubacterium rectale, Eubacterium hallii, and Roseburia species, the influence of other bacteria is likely insignificant and negligible, and any effect may be interpreted as the result of the influence of F. prausnitzii. The comparison results from the comparison of 12 pairs of samples revealed that samples with a low F06 level had higher amounts of butyrate and propionate than samples with a high F06 level (see FIGS. 6A and 6B). Such a great difference in the amounts of SCFAs is important data because the great difference suggests that the gut environment was very different between experimental group and control group. Differences other than the presence of AD, such as age, did not have a notable influence on the SCFA profile, and accordingly, it was concluded that the F06 level was the most important factor.

Because the L2-6 strain is related to the F06 clade and because the four other strains have the same butyrate biosynthesis pathway, the gene for the key enzyme butyryl CoA:acetate CoA-transferase was investigated to determine whether there was a difference in the expression of a pathway. The promoter of the above gene in each strain was synthesized and cloned into a promoter-probe vector upstream of the lacZ reporter gene, and gene expression was compared in E. coli (see FIGS. 6A and 6B). Although the above comparison was not performed in a F. prausnitzii host, the relative strengths of the promoters may be compared because the same conditions for the comparison were applied. The results show that the promoter of the A2-165 strain was significantly stronger than the promoters of the L2-6 strain and the other strains (see FIGS. 6A and 6B). In fact, the results are consistent with a study that reported a difference in butyrate production between the A2-165 and L2-6 strains. Moreover, the results indicate that butyrate levels in the samples were determined based on the composition of F. prausnitzii subspecies. Thus, dysbiosis of the F. prausnitzii associated with AD may result in the suppression of a high butyrate producer, such as A2-165 type bacteria, which may lead to a reduction in butyrate production.

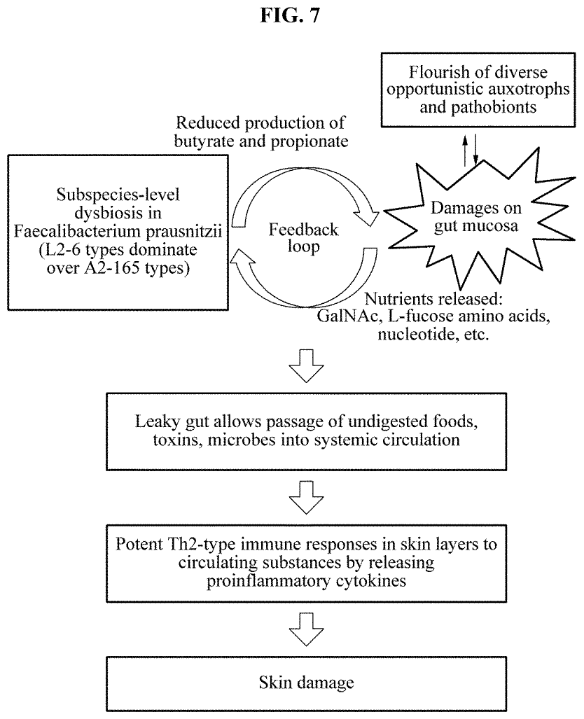

FIG. 7 illustrates a proposed model of AD associated with the dysfunction of F. prausnitzii.

F. prausnitzii, a dominant human intestinal bacterial species, has been shown to benefit gut health through the production of SCFAs. In particular, butyrate has an anti-inflammatory function and is used as a direct energy source by gut epidermal cells. Decreases in the level of F. prausnitzii and the production of SCFAs are associated with a Crohn disease. It has been reported that balance among subspecies of F. prausnitzii, in particular, changes between L2-6 and A2-165-type bacteria, affects the production of SCFAs. The AD metagenome was revealed to have an increased number of genes that utilize a mucin component, such as GalNAc and L-fucose, and various nutrients that may be released from damaged gut epithelial cells (see FIGS. 5A and 5B), which may indicate an increase in an inflammation in the gut epithelial cells and may correspond in a broad sense to a “leaky gut syndrome.” When gut epithelial cells are damaged because of a dysbiosis in F. prausnitzii or other causes, further dysbiosis in F. prausnitzii and the growth of a variety of auxotrophs and pathobionts may be simultaneously promoted. As a result, a feedback loop may be formed between the dysbiosis in F. prausnitzii and the dysregulation of a gut epithelial inflammation.

While this disclosure includes specific example embodiments, it will be apparent to one of ordinary skill in the art that various changes in form and details may be made in these example embodiments without departing from the spirit and scope of the claims and their equivalents. The example embodiments described herein are to be considered in a descriptive sense only, and not for purposes of limitation. Descriptions of features or aspects in each example embodiment are to be considered as being applicable to similar features or aspects in other example embodiments. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

1. A biomarker composition comprising a nucleotide sequence of SEQ ID NO: 1 and SEQ ID NO: 2 as an active ingredient.

2. The biomarker composition of claim 1, wherein the biomarker composition detects a subspecies of F. prausnitzii (F06).

3. The biomarker composition of claim 1, wherein the biomarker composition is used for diagnosis of atopic dermatitis (AD).

4. The biomarker composition of claim 1, wherein the biomarker composition comprises a nucleotide sequence having a sequence identity of 97% or higher to SEQ ID NO: 1 and SEQ ID NO: 2 as an active ingredient.

5. A diagnostic kit comprising the biomarker composition of claim 1.

6. A method of providing information, for predicting the possibility of atopic dermatitis or diagnosing atopic dermatitis comprising:
isolating a sample from a subject; and
detecting a subspecies (F06) of Faecalibacterium prausnitzii from the sample.

7. The method of claim 6, wherein the detection of the subspecies (F06) of F. prausnitzii is made by recognizing a nucleotide sequence including SEQ ID NO: 1 and/or SEQ ID NO: 2.

8. The method of claim 6, wherein the detection of the subspecies (F06) of F. prausnitzii is made by recognizing a nucleotide sequence including:
a nucleotide sequence which is at least 97% identical to SEQ ID NO: 1; and/or
a nucleotide sequence which is at least 97% identical to SEQ ID NO: 2.

9. The method of claim 6, wherein the detection of the subspecies (F06) of F. prausnitzii is made by recognizing a nucleotide sequence including:
a nucleotide sequence of SEQ ID NO:1 in which G at base position 9 is replaced with A, C at base position 13 is replaced with T, and/or A at base position 24 is replaced with G; and/or
a nucleotide sequence of SEQ ID NO:2 in which G at base position 3 is replaced with A, C at base position 5 is replaced with G, G at base position 16 is replaced with C, A at base position 20 is replaced with G, T at base position 32 is replaced with C, and/or G at base position 33 is replaced with A.

10. The method of claim 6, further comprising,
when a relative abundance of the subspecies (F06) of F. prausnitzii is higher than that of the normal group, determining the subject with atopic dermatitis or a high-risk group.

11. The method of claim 6, wherein the sample is at least one selected from stool, excreta, dejecta, stercus, feces and gut mucosa.
